(19) United States
(12) Patent Application Publication
(10) Pub. No.: US 2021/0161542 A1
(43) Pub. Date: Jun. 3, 2021

(54) ORTHOPEDIC DRILL ATTACHMENT FOR ALIGNMENT AND PENETRATION MEASUREMENT

(71) Applicant: Andrew Kafoury, Oxford, OH (US)

(72) Inventor: Andrew Kafoury, Oxford, OH (US)

(21) Appl. No.: 16/701,354

(22) Filed: Dec. 3, 2019

(51) Int. Cl. A61B 17/17; A61B 34/20; A61B 17/16; A61B 90/00

(52) U.S. Cl. CPC A61B 17/1707; A61B 34/20; A61B 2017/00022; A61B 90/06; A61B 17/1626

(57) ABSTRACT

Am electronic drill guidance system includes an arm unit and a drill unit. The arm unit is attachable to a C-Arm and the drill unit is attachable to a surgical drill. The arm unit sends target angular information to the drill unit. The drill unit determines whether the surgical drill is in an appropriate positon based on the received target information and measured angular position of the drill.

### CROSS-REFERENCE TO RELATED APPLICATION

This application claims priority to U.S. Provisional Patent Application No. 62/775,403 entitled “ORTHOPEDIC DRILL ATTACHMENT FOR ALIGNMENT AND PENETRATION MEASUREMENT,” filed on Dec. 5, 2018, which is incorporated herein by reference in its entirety.

### TECHNICAL FIELD

The present disclosure relates to an electronic drill guidance system and, more particularly, to an orthopedic drill attachment for alignment and penetration measurement.

### BACKGROUND

Orthopedic or surgical drills are used in performing surgical procedures. Surgical procedures may involve repairing bones, implanting devices, drilling cavities, replacing joints, removing undesirable material, or other operations. For example, a surgical drill may be used to drill pilot holes for surgical screws. The surgical screws may be used with plates or other devices to fix bones into position. Surgical drills are used by surgeons on a daily basis.

Surgeons must monitor the drill to make sure that the drill is properly aligned and positioned. As the drill moves, the surgeon must make sure that drill remains aligned and penetrates an appropriate depth without penetrating further. For instance, surgeons must caution so as to not to remove unnecessary material.

Even in common orthopedic surgeries, it is usual to see adverse effects when using traditional drilling systems. Some studies suggest that up to 47% of all patients who had undergone orthopedic surgery experienced at least one adverse event within 90 days.

Some traditional practices rely on surgeons to “eyeball” or estimate during operations. This often requires surgeons to guess or estimate the position of the drill and the depth of the drill head. Some mechanical guides can be used, but surgeons may find these guides too rigid or restrictive. As such, surgeons may rely too much on their own visual, tactile, and auditory estimation for the alignment and penetration of a drill when in the operating room. Accordingly, drilling bone with accuracy may require bulky and costly external equipment. The consequences for off-target drilling include reduced construct rigidity, premature loosening of the screws, and damage to the surrounding structures or tissue which can cause conditions such as avascular necrosis.

Therefore, a need exists for improved systems and methods for displaying information with a blending system and to modify the applicable pre-programmed blending operations.

### SUMMARY

Described herein is an electronic drill guidance system comprising a drill unit operatively attachable to a surgical drill, the drill unit comprising at least one sensor operatively measuring movement and positional information associated with the surgical drill; a communication component operatively communicatively coupled to at least one other device through a communication protocol to receive information; and an interface operatively generating at least one of audible or visual information based on the measured movement and positional information and information received via the communication component. The at least one sensor includes a digital motion processor. The digital motion processor determines angular data based on the movement and positional information. The movement and positional information includes at least one of raw angular information or acceleration. In an example, the at least one sensor includes a first sensor and a second sensor, wherein the first sensor and the second sensor comprise different types in comparison to each other. The first sensor comprises an accelerometer and the second sensor comprises a gyroscope. The drill unit may further include a filter operatively combining measurements from the first sensor and the second sensor. The arm unit may further include at least one of a Kalman filter or a Madgwick filter. The interface may comprise at least one of a display device or an audio device. The display device operatively generates a graphical depiction of an angular position of the surgical drill. The graphical depiction includes crosshairs and a target token, wherein the crosshairs depict a target orientation and the target token illustrates the angular position. The interface operatively generates a warning based on the measured movement and positional information and the information received via the communication component.

Also described is an electronic drill guidance system comprising an arm unit operatively attachable to an object, and comprising a first inertial sensor and a first communication component; and a drill unit operatively attachable to a surgical drill, and comprising a second inertial sensor and a second communication component, wherein the first inertial sensor measures angular information, and wherein the first communication device transmits target angular information based on the measured angular information, and wherein the second communication component receives the target angular information for comparison with measurements from the second inertial sensor. The drill unit displays a penetration measurement of a drill bit based on a comparison of readings from the arm unit and the drill unit via magnetic field sensing. The drill unit displays a torque measurement of a drill bit based on a comparison of readings from the arm unit and the drill unit via magneto-elastic sensing. The drill unit displays a depth measurement of a drill bit based on a comparison of readings from the arm unit and the drill unit.

Further described is a method for a drill guidance system comprising: providing an arm unit operatively attachable to a c-arm; providing a drill unit operatively attachable to a surgical drill; measuring, via the arm unit, angular information associated with the c-arm; measuring, via the drill unit, angular information associated with the surgical drill; and creating a target orientation for the surgical drill based on the angular information associated with the c-arm; generating a graphical display identifying the target orientation for the surgical drill and further identifying a current orientation of the surgical drill based on the angular information associated with the surgical drill. The method may further comprise providing at least two different types of sensors to measure angular information. The method may further comprise iterating the measuring, via the arm unit, angular information associated with the c-arm, and the measuring, via the drill unit, angular information associated with the surgical drill, to generate updated information in generally real-time. The method may further comprise generating notification to identify a potential error condition, wherein the error condition includes at least one of a misaligned bit or a bit having an improper size or shape.

### DESCRIPTION OF THE DRAWINGS

The accompanying drawings illustrate various systems, apparatuses, devices and methods, in which like reference characters refer to like parts throughout, and in which:

FIG. 1 is a functional block diagram of an electronic drill guidance system in accordance with various embodiments described herein;

FIG. 2 is a schematic of a drill unit in accordance with various embodiments described herein;

FIG. 3 is a schematic of an arm unit in accordance with various embodiments described herein;

FIG. 4 is a prototype of an arm unit without a housing in accordance with various embodiments described herein;

FIG. 5 is a prototype of a drill unit without a housing in accordance with various embodiments described herein;

FIG. 6 is an environmental diagram of an exemplary drill unit and drill in accordance with various embodiments disclosed herein;

FIG. 7 is a block diagram of a functional computer system in accordance with various embodiments described herein; and

FIG. 8 is an environmental diagram of the exemplary drill unit of FIG. 6, illustrating a magneto-electric sensor in accordance with various embodiments disclosed herein.

### DETAILED DESCRIPTION

Reference will now be made to exemplary embodiments, examples of which are illustrated in the accompanying drawings. It is to be understood that other embodiments may be utilized and structural and functional changes may be made. Moreover, features of the various embodiments may be combined or altered. As such, the following description is presented by way of illustration only and should not limit in any way the various alternatives and modifications that may be made to the illustrated embodiments. In this disclosure, numerous specific details provide a thorough understanding of the subject disclosure. It should be understood that aspects of this disclosure may be practiced with other embodiments not necessarily including all aspects described herein, etc.

As used herein, the words “example” and “exemplary” mean an instance, or illustration. The words “example” or “exemplary” do not indicate a key or preferred aspect or embodiment. The word “or” is intended to be inclusive rather an exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggests otherwise.

Moreover, terms such as “access point,” “server,” and the like, are utilized interchangeably, and refer to a network component or appliance that serves and receives control data, voice, video, sound, or other data-stream or signaling-stream. Data and signaling streams may be packetized or frame-based flows. Furthermore, the terms “user,” “customer,” “consumer,” and the like are employed interchangeably throughout the subject specification, unless context suggests otherwise or warrants a particular distinction among the terms. It is noted that such terms may refer to human entities or automated components supported through artificial intelligence (e.g., a capacity to make inference).

“Logic” refers to any information and/or data that may be applied to direct the operation of a processor. Logic may be formed from instruction signals stored in a memory (e.g., a non-transitory memory). Software is one example of logic. In another aspect, logic may include hardware, alone or in combination with software. For instance, logic may include digital and/or analog hardware circuits, such as hardware circuits comprising logical gates (e.g., AND, OR, XOR, NAND, NOR, and other logical operations). Furthermore, logic may be programmed and/or include aspects of various devices and is not limited to a single device.

Embodiments may utilize substantially any wired or wireless network. For instance, embodiments may utilize various radio access networks (RAN), e.g., Wi-Fi, global system for mobile communications, universal mobile telecommunications systems, worldwide interoperability for microwave access, enhanced general packet radio service, third generation partnership project long term evolution (3G LTE), fourth generation long term evolution (4G LTE), third generation partnership project 2, BLUETOOTH®, ultra mobile broadband, high speed packet access, x^{th} generation long term evolution, or another IEEE 802.XX technology. Furthermore, embodiments may utilize wired communications.

In traditional systems, surgeons typically rely either on their own estimation regarding drill depth or take additional images as the drill bit is inside the bone to discover penetration depth. Methods relying on a surgeon's experience or estimations are ineffective when manually driving a drill into bone. If a surgeon allows too much penetration, the patient can suffer serious damage to surrounding nerves, blood vessels, muscles, bone or other tissue.

There are many factors that will affect a surgeon's ability to properly drill, including drilling speed, axial drilling force, feed rate, and environmental distractions (e.g., noise). The difficulty in guiding a surgical drill is increased as the drill tip enters or drills through bone or other tissue as visual estimations become impaired. Further, different densities within the bone may also deflect the drill.

Some traditional systems use drill guides, which are bulky mechanical drill positioners. These guides, however, annoy surgeons with their size and frequent need for realigning which consumes time. Real-time ultrasound guidance is employed in fields like neurosurgery, but this practice is extremely limited in orthopedics because of the hardness and density of bones.

The angle at which a drill enters bone is critical. In some instances, determining a target trajectory angle requires precise imaging is obtained typically through a computed tomography (CT) scan. From the obtained imaging, the surgeon determines a safe surface entry point and trajectory angle so that a manually controlled drill can safely reach a target. Surgeons then guide drills by visual estimation. This is an imprecise method and can have serious drawbacks as described herein.

Additional CT scans may be implemented once the drill tip is inside the bone to determine its orientation. These extra imaging iterations greatly increase the length of the surgery. Fluoroscopy is a technique that may be employed to offer less timely x-ray imaging for depth. However, angulation of the drill is not measured and is still estimated by the user. These additional imaging techniques will increase the patient's exposure to ionizing radiation as well as drive up the cost of the surgery.

For example, C-Arm imaging utilizes an x-ray beam that passes from an emitter to a receiver. The C-Arm is positioned around a bone in such a way that models the desired direction for the drill as it enters the bone. Some mechanical devices may be used, such as radiopaque sights, to assist in alignment above the drill. However, mechanical sights often leave room for error and may be too cumbersome for some surgeons.

Others have attempted to use handheld devices like smartphones or iPOD TOUCH devices. The surgeon attempts to hold the handheld device with one hand and the drill with the other. Still other systems separate displays from surgical implements. This requires the surgeon to break a line of sight to view display. These attempts have suffered from poor line of sight, difficulty in operation, bulky setups, and unreliable results. Moreover, such handheld devices do not account well for tolerance or target angle, do not respond with any visual or auditory feedback, and do not measure depth of drilling.

Likewise, some systems use additional shafts or jigs protruding from surgical drills. These additional jigs may touch the bone to measure distance through a Linear Variable Differential Transformer (LVDT) or Rotary Variable Differential Transformer (RVDT). As with other attempts, these additional jigs are undesirable as it is another bulky member that alters the line of sight, weight, and usability of surgical drills. Moreover, such jigs often require specially designed drills and are unusable for existing drills. Further still, such jigs require a larger incision to allow room for the shaft to be placed against the bone. The area around the drill incision is likely complex with ridges and curves and may not be suitable to for jig placement.

Described embodiments provide for a dynamic, universal electronic drill guidance system comprising an orthopedic drill attachment for alignment and penetration measurement of a surgical drill. Embodiments may improve accuracy in drilling and may meet or exceed acceptable errors, such as of about 3° to 5° in alignment. Moreover, embodiments may seamlessly align a drill and measure the depth of a drill bit in bone all relative to a C-Arm or other device. It is noted that disclosed drill guidance systems may include one or more housings comprising wireless communication devices so that the system is wireless. Other embodiments, however, may include wired or wireless connections.

In examples, drill guidance systems may be operatively attached to mount on any desired surgical drill. For instance, a drill guidance system may include a housing that is removably attached to a surgical drill of any appropriate size, shape, make, or model. As such, surgeons can incorporate disclosed embodiments into existing drills to which they are familiar. This may reduce overall cost for surgeons, ease of use, and improved safety.

Disclosed embodiments may be implemented to train and assist surgeons while drilling, cutting, nailing, or otherwise conducting surgical procedures on bone. Methods may provide for aligning a drill according to a specified position, determining movement of the drill about an axis, measuring a depth of insertion of the drill, and providing notifications to a surgeon. Moreover, described methods may allow a surgeon to maintain the tool and interfaces in a line of sight.

The systems and methods described herein may increase precision and reduce errors in the operating room by directing the alignment of a drill or other medical device to a pre-positioned C-Arm imaging machine or other device. Embodiments may calculate offset of a drill in two or more axes (in degrees) relative to a C-Arm and may display navigational information via an interface. In examples, the navigational information may include instructions directing a user to move or position a drill in one or more directions for appropriate alignment. As such, there may not be a need for fluoroscopy.

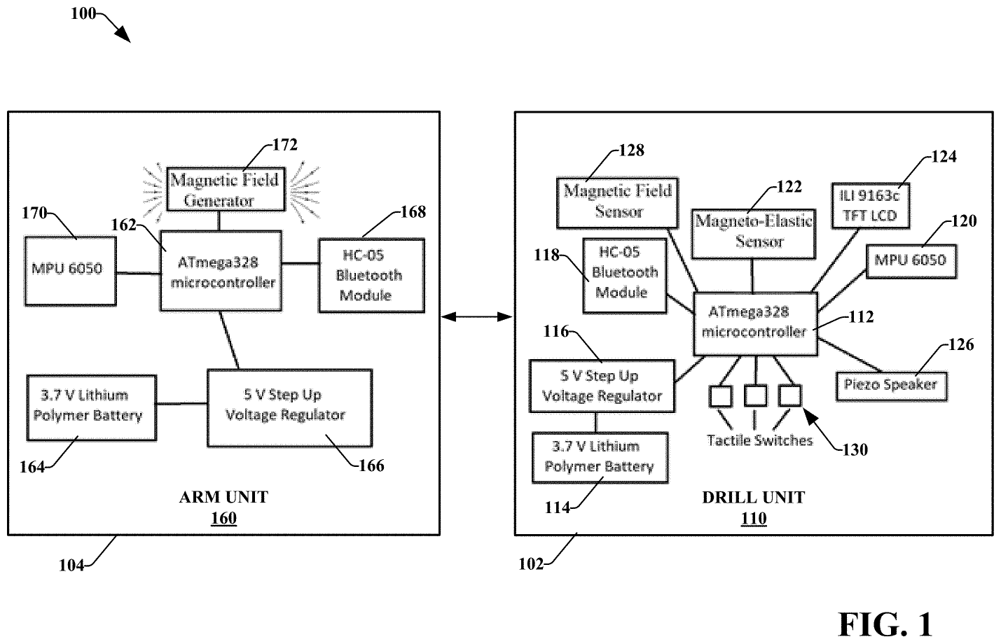

Referring now to FIG. 1, there depicted is a block diagram of a functional electronic drill guidance system 100 that may determine a target angle for a surgical drill and may generate guidance notifications for the surgical drill. The drill guidance system 100 may comprise a drill unit 110 and an arm unit 160. The drill unit 110 and the arm unit 160 may be communicatively coupled as described herein. The arm unit 160 may be attachable to an imaging device or other equipment in an operating room to measure angular and positional parameters. The arm unit 160 may communicate the measured parameters and may transmit them to the drill unit 110. The drill unit 110 may receive the parameters and may determine an orientation of the surgical drill and an insertion depth or relative distance between the drill unit 110 and the arm unit 160.

The arm unit 160 may comprise a housing 104. The housing 104 may house or enclose operative elements of the arm unit 160. In an aspect, the housing 104 may comprise any appropriate material, such as plastics, metals, or the like. It is noted that the housing 104 may be hermetically sealed such that the housing 104 may be submersible in fluid for disinfection. It is noted that the housing 104 may be any appropriate size and shape. For instance, the housing 104 may be sized to allow for attachment to a C-arm or other appropriate objects within a surgical room. The housing 104 may be attached to the surgical drill via a magnet, adhesive, snap fit, fasteners, or another securing mechanism may be used.

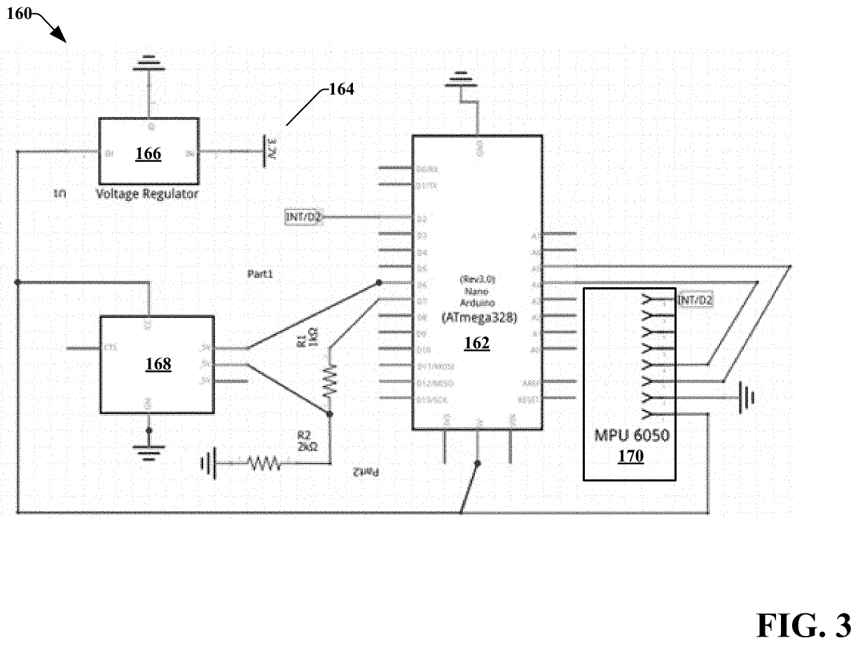

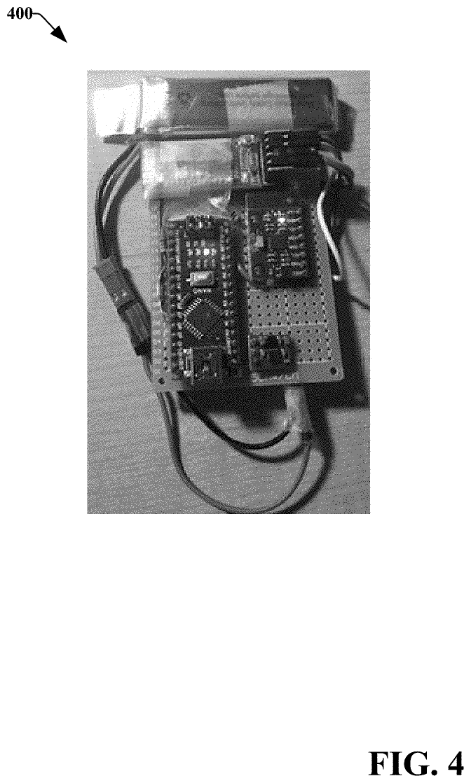

According to embodiments, the arm unit 160 may comprise a processor 162, a power source 164, a voltage regulator 166, a communication component 168, and an inertial sensor 170. It is noted that an exemplary schematic of the drill unit 110 is shown in FIG. 3. An exemplary prototype is shown in FIG. 4. Moreover, some embodiments may include a magnetic field generator 172, as described herein.

The processor 162 may comprise or communicate with a memory that may store computer executable instructions. The processor 162 may receive input from other components and may generate output, such as instructions, to the other components. For instance, the processor 162 may receive data from the inertial sensor 170 and may induce the communication component 168 to generate a wireless signal to be sent to the drill unit 110. In some embodiments, the processor 162 may comprise a low power device to minimize charging requirements of the power source 164, which may be a rechargeable and wireless power source (e.g., inductively charging power source). It is noted, however, that the power source 164 may be power mains or a removable or disposable power source.

As described herein, the arm unit 160 may be positionable on a C-arm. It is noted that the arm unit may be positioned anywhere on the C-arm, such as a face of the C-arm. In C-arm imaging, the C-Arm is positioned around a bone in such a way to model the direction the drill must enter the bone. For example, the C-Arm is aligned to the orientation in which the drill needs to cut through the bone and images are obtained relative to the C-Arm. The arm unit 160 mounts to the face of the C-Arm receiver and the inertial sensor 170 determines its angular data with respect to the transverse (pitch) axis and sagittal (roll) axis.

According to at least one embodiment, the inertial sensor 170 may comprise a Micro-Electro-Mechanical Systems (MEMS) device. The MEMS device may include, for example, a multi-axis sensor, such as a 6-axis gyroscope and accelerometer sensor. In an example, the accelerometer may comprise a multi-axis (e.g., two, three, etc.) accelerometer, and the gyroscope may comprise a multi-axis (e.g., two, three, etc.) gyroscope. The sensitive axis of the accelerometer and gyroscope may measure raw gravitational parameters, positional data, angular data, movement, and the like. In some embodiments the inertial sensor 170 may include an on-board processor, such as a digital motion processor (DMP). It is noted, however, that the inertial sensor 170 may not comprise a processor and may communicate information directly to the processor 162. In either case, the on-board processor or the processor 162 may determine angular data for alignment of a drill based on the raw angular data received from the accelerometer and/or gyroscope. It is further noted that the arm unit 160 may include filters within inertial sensor 170 or otherwise disposed between the inertial sensor and the processor 162. As an example, a filter may be utilized to combine accelerometer and gyroscope readings, which may reduce drawbacks of a single type of sensor. Other filters may be utilized, such as a Kalman filter, Madgwick filter, or the like.

The angular data may be received by the communication component 168 which may transmit the angular data. In some examples, the communication component 168 may comprise communication drivers, transceivers/receivers (e.g., Wi-Fi, NFC, or the like), USB drives, or other components that may communicate with other devices. For example, the communication component 168 may comprise a BLUETOOTH device that operatively communicates with the drill unit 110 or other devices such as a user device, or a smart phone running a mobile application.

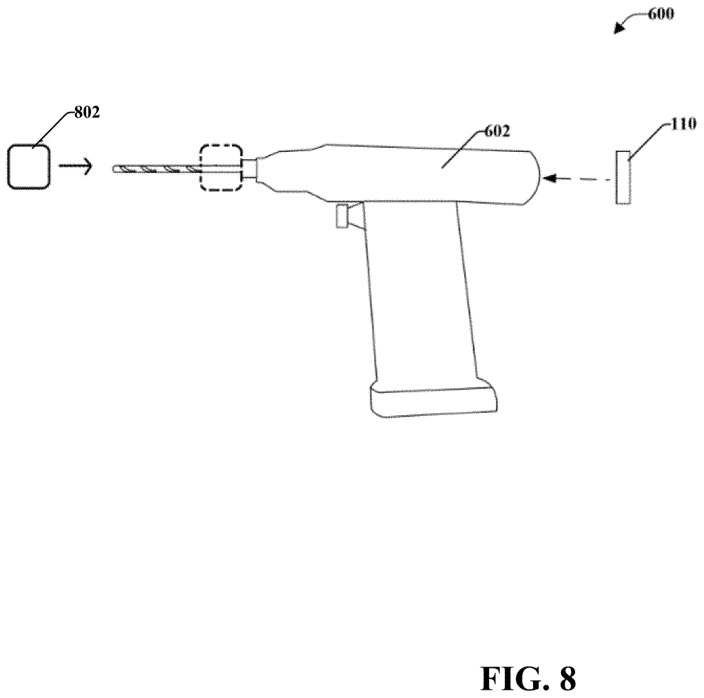

According to at least some embodiments, the arm unit 160 may comprise position tracking devices that measure absolute positon relative to two or more objects as opposed to angular data described above. For instance, the arm unit 160 and the drill unit 110 may utilize sensors or devices to measure relative positions. It is noted that the position tracking devices may comprise optical sensors (e.g., cameras that identify objects), audio sensors, or the like. In an exemplary embodiment, the system 100 may comprise a magnetic field generator 172 disposed within the arm unit 160. The magnetic field generator 172 may transmit or emit a magnetic field. The magnetic field may be measured by a magnetic field sensor or magneto-elastic sensor disposed in a drill unit 110 as described herein. It is noted, however, that the arm unit 160 may comprise a magnetic field sensor or magneto-elastic sensor and the drill unit 110 may comprise a magnetic field generator. The magneto-elastic sensor 802 may be disposed in any appropriate location, such as disposed on or about a drill bit as depicted in in FIG. 8. It is noted that that drill bits may be magnetically conditioned to produce a circumferential magnetic field within the shaft to be picked up by the magneto-elastic sensor 802.

In examples where the arm unit 160 comprises the magnetic field generator 172, the drill unit 110 comprises a magnetic field sensor 120 that may measure the change in magnetic flux. The arm unit 160, such as via processor 112, can resolve the output of the magnetic field sensor 120 into a distance from the transmitter. Since the rate of change in a generated magnetic field is measured, static magnetic fields are not measured (e.g. Earth's magnetic field). The magnetic field generated by the magnetic field generator 172 is far below the threshold of what can create problems for other devices in the operating room, such as pacemakers. It is noted that embodiments may utilize other methods and devices that may wirelessly determine positional information. In some embodiments, the drill unit 110 may be zeroed or otherwise calibrated when in place for an operation. This may give the system 100 a reference point for determining cut depth during operation, which may be utilized to determine whether the drill has reached a target cut depth that may be predetermined and entered by a user or other device.

In embodiments, the drill unit 110 may comprise a housing 102. The housing 102 may house or enclose operative elements of the drill unit 110. In an aspect, the housing 102 may comprise any appropriate material, such as plastics, metals, or the like. It is noted that the housing 102 may be hermetically sealed such that the housing 102 may be submersible in fluid for disinfection. It is noted that the housing 102 may be any appropriate size and shape. For instance, the housing 102 may be sized to allow for attachment to a surgical drill. The housing 102 may be attached to the surgical drill via a magnet, adhesive, snap fit, fasteners, or another securing mechanism may be used.

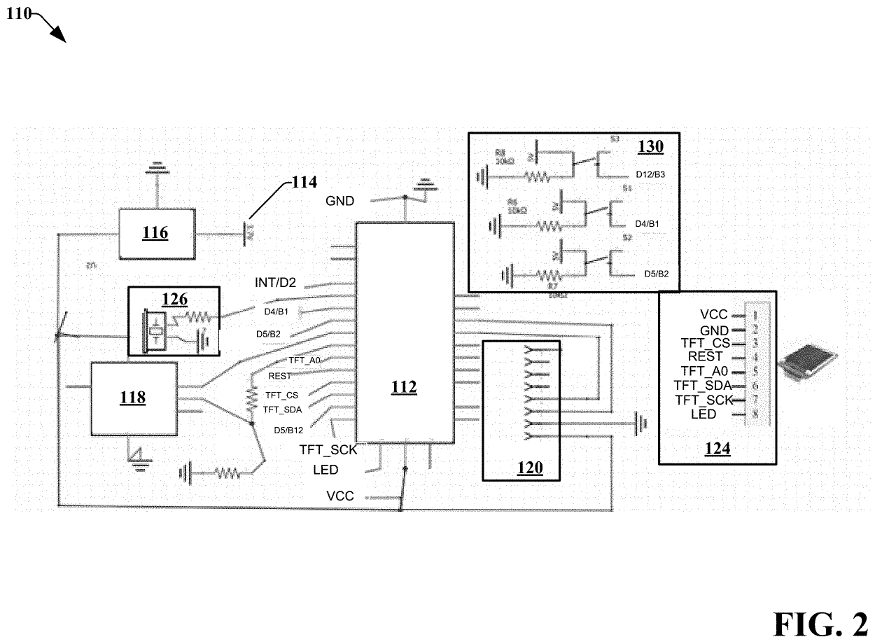

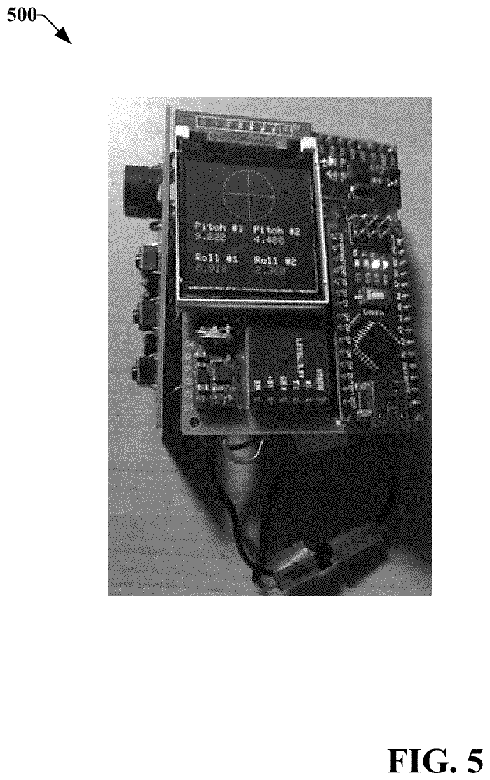

The operative elements of the drill unit 110 may primarily comprise a processor 112, a power source 114, a voltage regulator 116, a communication component 118, an inertial sensor 120, user interface device(s) (e.g., display device 124, audio device 126, input devices 130, etc.). In at least some embodiments, the drill unit 110 may include positon sensors as described herein. It is noted that an exemplary schematic of the drill unit 110 is shown in FIG. 2. An exemplary prototype is shown in FIG. 5. The processor 112 may comprise or communicate with a memory that may store computer executable instructions. The processor 112 may receive input from other components and may generate output, such as instructions, to the other components. For instance, the processor 112 may control the display device 124, audio device 126, and other components.

In described embodiments, the drill unit 110 may receive, via the communication component 118, angular data or position data from the communication component 168 of the arm unit 160. The communication component 118 may comprise communication drivers, transceivers/receivers (e.g., Wi-Fi, NFC, or the like), USB drives, or other components that may communicate with other devices. For example, the communication component 118 may comprise a BLUETOOTH device that operatively communicates with the communication component 168 of the arm unit 160 or other devices such as a user device, or a smart phone running a mobile application.

The processor 112 may analyze the received orientation information and may treat the orientation data as a target or reference orientation. The processor 112 may utilize the reference orientation along with angular orientation data received from the inertial sensor 120. It is noted that the inertial sensor 120 may comprise a similar or identical make as the inertial sensor 170 of the arm unit 160. This may allow the processor 112 to compare measurements received from the inertial sensor 120 with measurements from the inertial sensor 170. In some embodiments, the processor 112 may compare instantaneous measurements or may compare a history of measurements (e.g., an average of the ten most recent measurements or the like) to filter out erroneous readings. The comparison may allow the processor 112 to determine whether the drill is appropriately oriented, whether the position of the drill needs adjusted, and appropriate adjustments to align the drill.

The drill unit 110 may generate notifications to a user regarding the drill orientation relative to the target orientation. For instance, the processor 112 may send instructions to the display device 124 or audio device 126 that cause the devices to render visual information or audio notifications. For instance, the processor 112 may instruct the display device 124 or audio device 126 to generate warning notifications if the drill is outside a specified range (e.g., 3 degree variance, etc.) relative to a target angle. It is noted that the display device 124 may comprise an LCD display, LED's, 7-segment displays, or the like.

As described herein, the drill unit 110 receives angular data from the arm unit 160, such as pitch and roll values of the C-Arm. The display device 124 may display the received values of the C-Arm and measured pitch and roll values of the drill unit. It is noted that the display device 124 may iterate updates to display on a real-time or near real-time basis. In at least some embodiments, the display device 124 may generate graphical depictions of the angular position. For instance, the display device 124 may generate crosshairs and a target token (e.g., circle, dot, etc.) wherein the crosshairs depict the target orientation and the target token illustrates the drill orientation. For example, if the user tilts the drill to the right (positive roll), the circle will move right along the x (roll) axis and so forth. When the drill is within the acceptable error for pitch or roll, the display device 124 may notify the user, such as through flashing, color display, textual identification, or the like. In another aspect, if the drill is not within the acceptable error, the display device 124 will generate a visual notification.

As described herein, the processor 112 may control the display device 124 and may analyze received data from the various sensors. In examples, the processor may transform angular data into an attitude and heading reference system (AHRS) that makes it very easy for the user to understand the position of the drill relative to the target angle.

In embodiments, the processor 112 controls audio device 126 to generate notifications to the user. For instance, the audio device 126 may generate voice data or sounds of different frequencies and tones for different durations to identify whether the drill is within an acceptable position, is not within an acceptable position, and/or to identify the severity of the drill position. For example, when the error in pitch and roll exceeds 45 degrees, rapid high-pitched noises are produced. When that error is reduced, less rapid and lower pitches are produced. When both pitch and roll are aligned, short, continuous beeps are heard. An RGB LED is configured to the top of the drill unit so it remains in a surgeon's field of view without obstructing vision. The RGB LED changes color as well with respect to the amount of error between the drill and C-Arm. A bar graph may be displayed next to the crosshairs to show the linear position of the drill.

In embodiments, the drill unit 110 may comprise input devices 130. Input device 130 may comprise tactical buttons, a touch screen, or the like. In an example, the drill unit 110 may comprise one or more buttons, such as three buttons that allow the user to set the tolerance for the drill. By pressing one of the buttons, the user can navigate through display screens and provide information to the drill unit 110.

In at least one embodiment, the drill unit 110 can include or communicate with a magneto-elastic sensor 122. The magneto-elastic sensor 122 may be disposed around a drill bit without making contact with the bit. As the drill bit undergoes constant force and torque while in use, the magnetic field of the steel bit is altered. These changes are small enough so as not to affect the magnetic sensor for linear position. This is then measured by the magneto-elastic sensor 122 and processed into torque and load readings. Bones are made up of different sections with different densities and material properties. Employing this sensor alerts surgeons when entering new bone material based on the changes of torque and load. It also provides real time feedback to surgeons regarding torque and load, which can be one of the biggest factors to preventing thermal necrosis. In another example, the sensor may detect unwanted movements and generated a signal that triggers an alarm or interface to alter a user. For instance, a magneto-elastic sensor may detect drill bit movement, which may be caused by a misaligned bit, a bit having an improper size or shape, or the like.

As described herein, embodiments may allow for alignment and penetration readings based off the position of a C-Arm. There is no need to input a target angle or use any additional effort to know the drill alignment and depth. For the first time, a C-Arm has direct communication and linkage to a drill. It is important to note that this device operates completely wirelessly and independent from additional computers and/or software. It allows the surgeon to receive every important piece of feedback directly on the back of the screen while maintaining the line of sight. Surgeons will no longer have to position themselves awkwardly or constantly move to view a computer. It also incorporates a magneto-elastic sensor to determine force and torque. This is new in the medical field and provides an advantage over existing sensors as it incorporates both torque and force. It can be attached and detached over the drill bit quickly and easily and never contacts the drill bit during operation. The feedback generated from the device not only gives the surgeon information about the alignment and linear position of the drill, but, with the magneto-elastic sensor, also provides data which can help identify different bone material and possible thermal necrosis. With the onboard screen, RGB LED, and speaker, the device produces an auditory response as well as both a detailed and general visual response. The device is fully enclosed so it is sterilizable. Its small size allows the surgeon to always maintain their line of sight while holding the drill as they would normally.

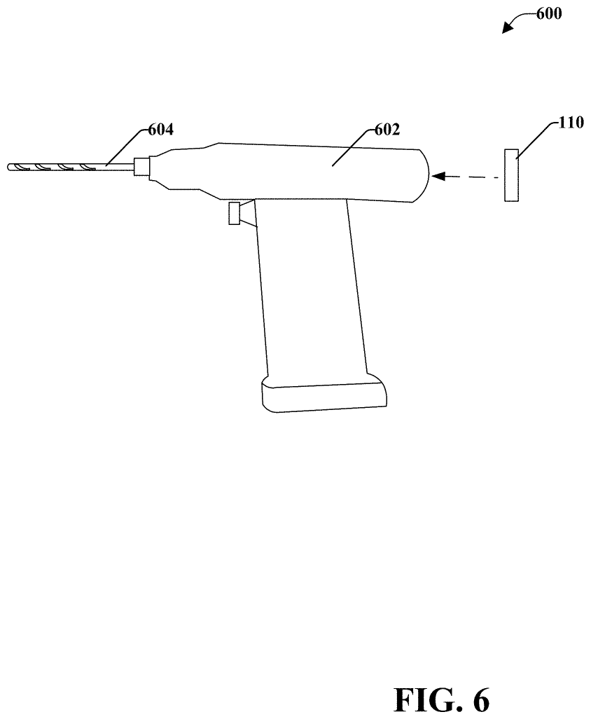

FIG. 6 illustrates and exemplary system 600 comprising drill 602, drill bit 604, and the drill unit 110. As illustrated, the drill unit 110 may be attached to the drill 602 at a location that is generally convenient for a surgeon. As shown, the surgeon may be free to grasp a handle and may have easy viewing access to the drill unit.

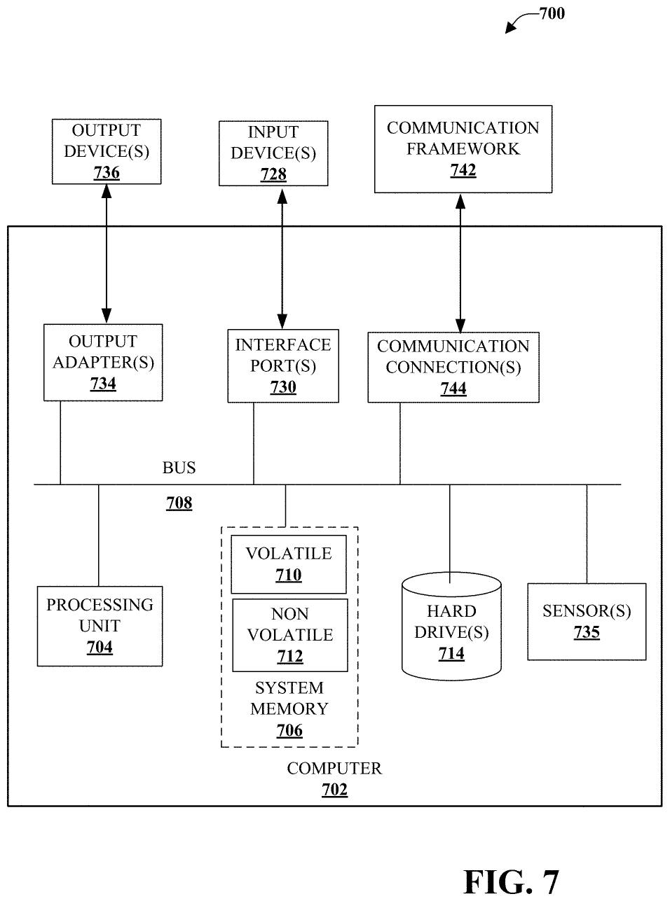

What has been described above may be further understood with reference to the following figures. FIG. 7 provides an exemplary operating environment or systems capable of implementing one or more systems, apparatuses, or processes described above. FIG. 7 is not intended to limit the scope of such systems, apparatuses, or processes.

FIG. 7 is a block diagram of a computer system 700 that may be employed to execute various disclosed embodiments. It is noted that various components may be implemented in combination with computer executable instructions, hardware devices, and/or combinations of hardware and software devices that may be performed by computer system 700.

Computer system 700 may include various components, hardware devices, software, software in execution, and the like. In embodiments, computer system 700 may include computer 700. Computer 700 may include a system bus 708 that couples various system components. Such components may include a processing unit(s) 704, system memory device(s) 706, disk storage device(s) 714, sensor(s) 735, output adapter(s) 734, interface port(s) 730, and communication connection(s) 744. One or more of the various components may be employed to perform aspects or embodiments disclosed herein. \

Processing unit(s) 704 may comprise various hardware processing devices, such as single core or multi-core processing devices. Moreover, processing unit(s) 704 may refer to a “processor,” “controller,” “computing processing unit (CPU),” or the like. Such terms generally relate to a hardware device. Additionally, processing unit(s) 704 may include an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or the like.

System memory 706 may include one or more types of memory, such as volatile memory 710 (e.g., random access memory (RAM)) and non-volatile memory 712 (e.g., read-only memory (ROM)). ROM may include erasable programmable ROM (EPROM), and/or electrically erasable programmable ROM (EEPROM). In various embodiments, processing unit(s) 704 may execute computer executable instructions stored in system memory 706, such as operating system instructions and the like.

Computer 702 may also contain one or more hard drive(s) 714 (e.g., EIDE, SATA). While hard drive(s) 714 are depicted as internal to computer 702, it is noted that hard drive(s) 714 may be external and/or coupled to computer 702 via remote connections. Moreover, input port(s) 730 may include interfaces for coupling to input device(s) 728, such as disk drives. Disk drives may include components configured to receive, read and/or write to various types of memory devices, such as magnetic disks, optical disks (e.g., compact disks and/or other optical media), flash memory, zip drives, magnetic tapes, and the like.

It is noted that hard drive(s) 714 and/or other disk drives (or non-transitory memory devices in general) may store data and/or computer-executable instructions according to various described embodiments. Such memory devices may also include computer-executable instructions associated with various other programs or modules. For instance, hard drives(s) 714 may include operating system modules, application program modules, and the like. Moreover, aspects disclosed herein are not limited to a particular operating system, such as a commercially available operating system.

Input device(s) 728 may also include various user interface devices or other input devices, such as sensors (e.g., microphones, pressure sensors, light sensors, etc.), scales, cameras, scanners, facsimile machines, and the like. A user interface device may generate instructions associated with user commands. Such instructions may be received by computer 702. Examples of such interface devices include a keyboard, mouse (e.g., pointing device), joystick, remote controller, gaming controller, touch screen, stylus, and the like. Input port(s) 730 may provide connections for the input device(s) 728, such as via universal serial ports USB ports), infrared (IR) sensors, serial ports, parallel ports, wireless connections, specialized ports, and the like.

Output adapter(s) 734 may include various devices and/or programs that interface with output device(s) 736. Such output device(s) 736 may include LEDs, computer monitors, touch screens, televisions, projectors, audio devices, printing devices, or the like.

In embodiments, computer 702 may be utilized as a client and/or a server device. As such, computer 702 may include communication connection(s) 744 for connecting to a communication framework 742. Communication connection(s) 744 may include devices or components capable of connecting to a network. For instance, communication connection(s) 744 may include cellular antennas, wireless antennas, wired connections, and the like. Such communication connection(s) 744 may connect to networks via communication framework 742. The networks may include wide area networks, local area networks, facility or enterprise wide networks (e.g., intranet), global networks (e.g., Internet), satellite networks, and the like. Some examples of wireless networks include Wi-Fi, Wi-Fi direct, BLUETOOTH™, Zigbee, and other 702.XX wireless technologies. It is noted that communication framework 742 may include multiple networks connected together. For instance, a Wi-Fi network may be connected to a wired Ethernet network.

The terms “component,” “module,” “system,” “interface,” “platform,” “service,” “framework,” “connector,” “controller,” or the like are generally intended to refer to a computer-related entity. Such terms may refer to at least one of hardware, software, or software in execution. For example, a component may include a computer-process running on a processor, a processor, a device, a process, a computer thread, or the like. In another aspect, such terms may include both an application running on a processor and a processor. Moreover, such terms may be localized to one computer and/or may be distributed across multiple computers.

What has been described above includes examples of the present specification. It is, of course, not possible to describe every conceivable combination of components or methodologies for purposes of describing the present specification, but one of ordinary skill in the art may recognize that many further combinations and permutations of the present specification are possible. Each of the components described above may be combined or added together in any permutation to define the blending system 100. Accordingly, the present specification is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An electronic drill guidance system comprising:
a drill unit operatively attachable to a surgical drill, the drill unit comprising:
at least one sensor operatively measuring movement and positional information associated with the surgical drill;
a communication component operatively communicatively coupled to at least one other device through a communication protocol to receive information;
and an interface operatively generating at least one of audible or visual information based on the measured movement and positional information and information received via the communication component.

2. The electronic drill guidance system of claim 1, wherein the at least one sensor includes a digital motion processor.

3. The electronic drill guidance system of claim 2, wherein the digital motion processor determines angular data based on the movement and positional information.

4. The electronic drill guidance system of claim 3, wherein the movement and positional information includes at least one of raw angular information or acceleration.

5. The electronic drill guidance system of claim 1, wherein the at least one sensor includes a first sensor and a second sensor, wherein the first sensor and the second sensor comprise different types in comparison to each other.

6. The electronic drill guidance system of claim 5, wherein the first sensor comprises an accelerometer and the second sensor comprises a gyroscope.

7. The electronic drill guidance system of claim 5, wherein the drill unit further includes a filter operatively combining measurements from the first sensor and the second sensor.

8. The electronic drill guidance system of claim 1, wherein the arm unit further comprises at least one of a Kalman filter or a Madgwick filter.

9. The electronic drill guidance system of claim 1, wherein the interface comprises at least one of a display device or an audio device.

10. The electronic drill guidance system of claim 9, wherein the display device operatively generates a graphical depiction of an angular position of the surgical drill.

11. The electronic drill guidance system of claim 10, wherein the graphical depiction includes crosshairs and a target token, wherein the crosshairs depict a target orientation and the target token illustrates the angular position.

12. The electronic drill guidance system of claim 9, wherein the interface operatively generates a warning based on the measured movement and positional information and the information received via the communication component.

13. An electronic drill guidance system comprising:
an arm unit operatively attachable to an object, and comprising a first inertial sensor and a first communication component; and
a drill unit operatively attachable to a surgical drill, and comprising a second inertial sensor and a second communication component,
wherein the first inertial sensor measures angular information, and wherein the first communication device transmits target angular information based on the measured angular information, and
wherein the second communication component receives the target angular information for comparison with measurements from the second inertial sensor.

14. The electronic drill guidance system of claim 10, wherein the drill unit displays a penetration measurement of a drill bit based on a comparison of readings from the arm unit and the drill unit via magnetic field sensing.

15. The electronic drill guidance system of claim 10, wherein the drill unit displays a torque measurement of a drill bit based on a comparison of readings from the arm unit and the drill unit via magneto-elastic sensing.

16. The electronic drill guidance system of claim 10, wherein the drill unit displays a depth measurement of a drill bit based on a comparison of readings from the arm unit and the drill unit.

17. A method for a drill guidance system comprising:
providing an arm unit operatively attachable to a c-arm;
providing a drill unit operatively attachable to a surgical drill;
measuring, via the arm unit, angular information associated with the c-arm;
measuring, via the drill unit, angular information associated with the surgical drill; and
creating a target orientation for the surgical drill based on the angular information associated with the c-arm;
generating a graphical display identifying the target orientation for the surgical drill and further identifying a current orientation of the surgical drill based on the angular information associated with the surgical drill.

18. The method of claim 17, further comprising, providing at least two different types of sensors to measure angular information.

19. The method of claim 17, further comprising iterating the measuring, via the arm unit, angular information associated with the c-arm, and the measuring, via the drill unit, angular information associated with the surgical drill, to generate updated information in generally real-time.

20. The method of claim 17, further comprising generating notification to identify a potential error condition, wherein the error condition includes at least one of a misaligned bit or a bit having an improper size or shape.
